(19) United States
(12) Patent Application Publication
(10) Pub. No.: US 2003/0147772 A1
(43) Pub. Date: Aug. 7, 2003

(54) Autoclave construction

(76) Inventor: Tari Taricco, Rancho Palos Verdes, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/355,427

(22) Filed: Jan. 31, 2003

Related U.S. Application Data
(60) Provisional application No. 60/354,401, filed on Feb. 4, 2002.

(51) Int. Cl. A61L 2/00; A61L 2/08; A61L 2/04

(52) U.S. Cl. 422/26; 422/295; 422/307

(57) ABSTRACT

Autoclave construction and methods that can substantially reduce shipping costs for large autoclaves. In accordance with the invention, the autoclave body, the door and the hinge assembly for mounting the door to the autoclave body are manufactured as bolt together assemblies, with the autoclave body shipped without the door or hinge assembly attached thereto. This reduces the size and weight of the autoclave, reducing its shipping costs much more than the cost of shipping the door and hinge assembly, whether assembled together or shipped as two separate parts. Alternatively, the door may be welded to the hinge assembly and shipped as one part, the hinge assembly and door being readily assembled to the autoclave body at the destination by bolting the two parts together.

### CROSS-REFERENCE TO RELATED APPLICATION

This application claims the benefit of U.S. Provisional Patent Application No. 60/354,401 filed Feb. 4, 2002.

### BACKGROUND OF THE INVENTION

1. Field of the Invention

The present invention relates to the field of autoclaves.

2. Prior Art

Autoclaves of all sizes are well known in the prior art. Of particular importance to the present invention are large autoclaves, where shipping size and/or weight presents a special problem. Because autoclaves comprise a pressure vessel, their manufacture requires special knowledge, skill, equipment and testing procedures preventing their construction at the location of use using ordinary steel fabrication techniques. Instead, autoclaves of any size are normally manufactured by an autoclave manufacturer and shipped to their location of use.

For larger autoclaves, it is common to weld the hinge assembly to the autoclave body and to the door to preserve alignment of the door relative to the autoclave body. Welding has the advantage of allowing the parts to be properly aligned, and then the alignment “captured” by welding to make the alignment permanent.

For large autoclaves, shipment of an autoclave can present special problems, and can be very expensive. Either or both size and weight can be troublesome. By way of example, an autoclave body may have an outer diameter approaching the limit for width of a normal truckload on the highway. Since normally the door of the autoclave needs to swing forward somewhat when opening, the door hinge axis is normally displaced somewhat outward from the side of the autoclave. This increases the width of the autoclave, making shipment on the highways more expensive. Similar problems are encountered when shipping using other modes of transportation. Shipping of larger autoclaves is even more troublesome.

Also as the size of an autoclave increases, the weight of the autoclave increases more rapidly. By way of example, doubling-the diameter and length of an autoclave will increase its weight by a factor of four, but then the larger autoclave will only take one half the pressure of the smaller autoclave. If the same pressure capability is to be maintained, then the wall thickness must be doubled, so that the larger autoclave will have 8 times the weight of the smaller one. Consequently shipping weight can also rapidly become a serious problem for the larger autoclaves.

### BRIEF SUMMARY OF THE INVENTION

Autoclave construction and methods that can substantially reduce shipping costs for large autoclaves are disclosed. In accordance with the invention, the autoclave body, the door and the hinge assembly for mounting the door to the autoclave body are manufactured as bolt together assemblies, with the autoclave body shipped without the door or hinge assembly attached thereto. This reduces the size and weight of the autoclave body, reducing its shipping costs much more than the cost of shipping the door and hinge assembly, whether assembled together or shipped as two separate parts. Alternatively, the door may be welded to the hinge assembly and shipped as one part, the hinge assembly and door being readily assembled to the autoclave body at the destination by bolting the two parts together.

### BRIEF DESCRIPTION OF THE DRAWINGS

FIG. 1 is a perspective view of the one embodiment of the present invention autoclave.

FIG. 2 is a side view of the forward region of the autoclave of FIG. 1.

FIG. 3 is a perspective exploded view of the autoclave of FIG. 1 illustrating the bolt together structure thereof.

### DETAILED DESCRIPTION OF THE PREFERRED EMBODIMENT

The present invention comprises autoclaves with a unique door and hinge assembly allowing the autoclave to be shipped completely assembled, shipped with the door and hinge assembly as a item separate from the autoclave body, or shipped with the door, the hinge assembly and the autoclave body as three separate items which may be readily assembled at the ultimate site using simple bolt-on procedures. The invention is particularly useful for large autoclaves and/or particularly heavy autoclaves where the removable door and hinge assembly reduces both the shipping size and weight of the main autoclave body, which can significantly reduce shipping costs. The invention is particularly applicable to autoclaves of an outer diameter of 6 feet or larger, and/or weighing 3000 pounds or more.

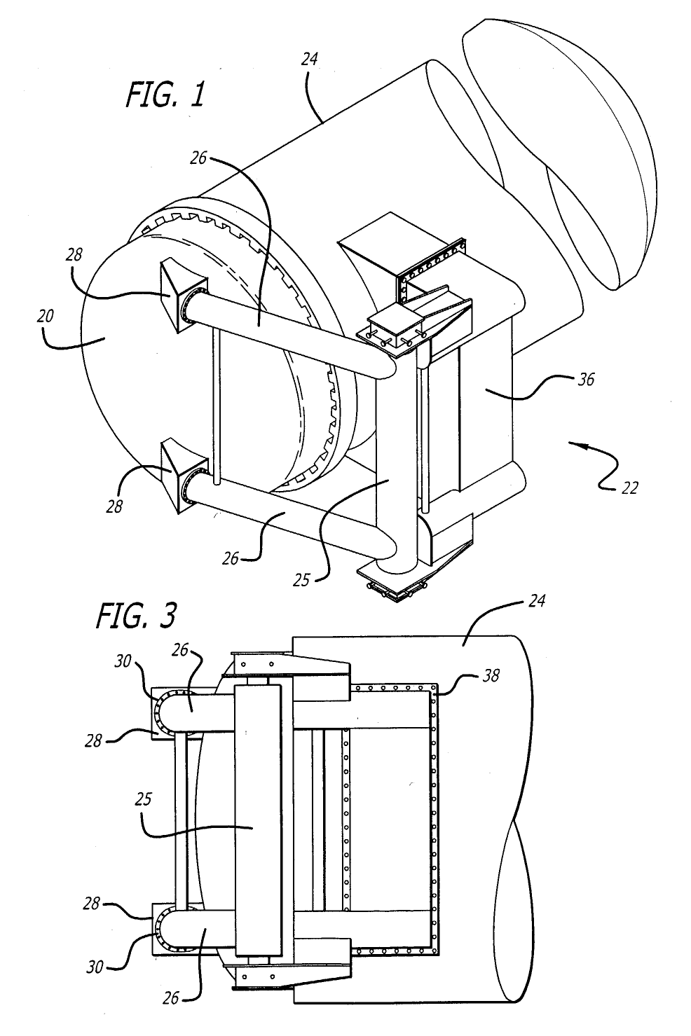

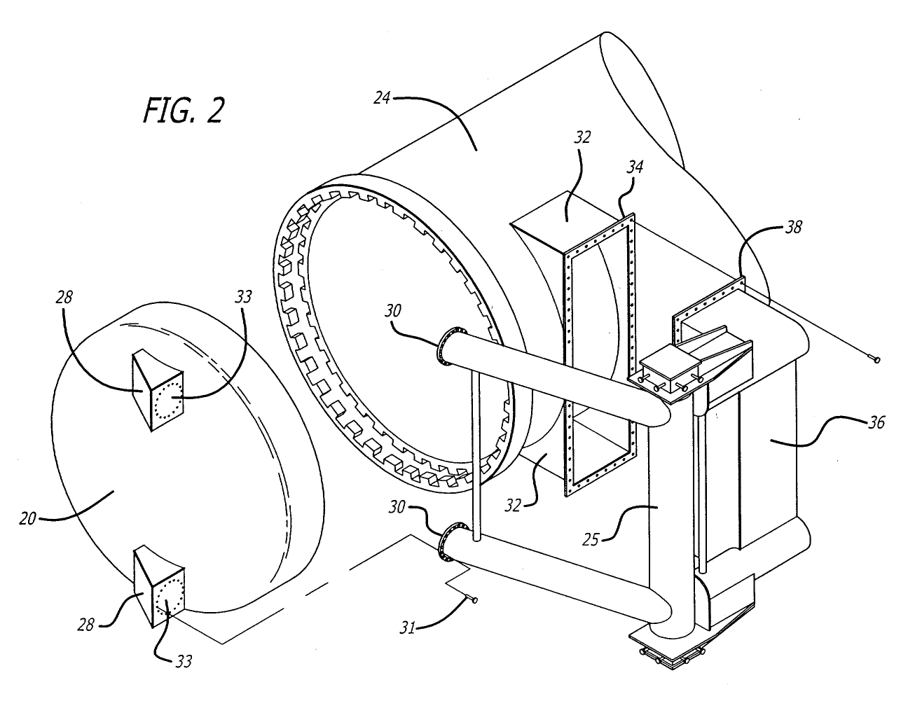

FIG. 1 shows an assembled autoclave in accordance with the present invention. The three major subassemblies important to the present invention are the front door 20, a hinge assembly generally indicated by the numeral 22 and the main body 24 of the autoclave. The hinge bearings, etc. may be in accordance with the prior art, and need not be described further herein. In accordance with one aspect of the present invention, and unlike the prior art, the arms 26 on the hinge assembly are bolted to box-like protrusions 28, which in turn are permanently welded to the door 20. The bolting of the arms 26 to the protrusions 28 may be better seen in FIG. 3, which Figure shows the bolting of the arms to the box-like structures 28 on the door. In particular, the arms 26 in this exemplary embodiment have flanges 30 welded thereto (best visible in FIG. 3) with a plurality of bolts 31 extending through the flanges and the cooperatively disposed faces 33 of the box-like protrusions 28. The bolts used to bolt the arms to the box-like protrusions may work in conjunction with ordinary nuts if access to the interior of the box-like protrusions 28 is provided, or as one alternative, nuts or other threaded members may be welded to the back of the plate on the box-like protrusions so that access to the interior of the box-like protrusions is not required during assembly of the arms to the door, or the plates themselves could be threaded if desired. Closed box-like protrusions and structures have an advantage of reducing corrosion by not collecting moisture, and of providing a neater, cleaner structure for maintenance purposes.

The hinge assembly and the manner of attachment thereof to the body of the autoclave may be seen in FIGS. 1 through 3. In FIG. 3, the hinge assembly 22 is shown in an exploded view, as if the hinge assembly is suspended from a crane for assembly purposes, being moved toward its assembled position. Also visible in FIG. 3 is a box-like structure 32 welded to the curved side of the autoclave body 24. The box-like structure is essentially an open box-like structure, in that it has an outward facing opening circumscribed by a flange 34. Hinge assembly 22 has a mating box-like structure 36, also circumscribed by a flange region 38. Flanges 34 and 38 have a plurality of cooperatively disposed bolt holes therein so that the flanges may be bolted together to fasten the hinge assembly to the autoclave body for assembly purposes. Because of the access to both sides of the flanges, conventional nuts and bolts may be used, or bolting alternatives such as those previously described may be used.

It will be noted from FIG. 2 that the box-like structure is welded to the autoclave body 24 so that the plane of flange 34 may be very close to, or even tangent to the side of the autoclave body 24, even though the rest of the hinge assembly, including the hinge axis (in the center of tube 25) typically will be much further from the axis of the autoclave body to allow the door to swing somewhat forward on opening to clear the door locking mechanism (which may be of conventional design). Thus the autoclave body 24 with the hinge mounting structure thereon is considerably narrower than the assembled autoclave, and also considerably lighter because of the absence of the hinge assembly, itself a substantial structure, and the door, which is part of the pressure vessel.

In FIGS. 1 and 3, the box-like structure 36 on the hinge assembly 22 is shown in its assembled position on the hinge attachment structure on the autoclave body, more specifically, with the flanges 34 and 38 mating and being bolted together. If desired, special alignment pins may be used to more accurately align the parts than is normally achieved by bolts through bolt holes.

FIGS. 1 and 3 show the flanges 34 and 38 bolted together by a relatively large plurality of bolts to positively assemble the hinge assembly to the autoclave body, and the arms 26 bolted to the door 20 by a relatively large plurality of bolts to positively assemble the door to the hinge assembly (here too, alignment pins or other special provision for alignment of the parts may be provided, if desired). This is to be compared with the prior art wherein the door is welded to the hinge assembly and the hinge assembly is welded directly to the autoclave, with the autoclave being shipped in the assembled condition.

With a bolt together autoclave assembly such as the exemplary embodiment disclosed herein, the autoclave body may be shipped as one piece or unit, and the door and hinge assembly shipped as another assembly, or the door and hinge assembly shipped at two additional pieces. (Alternatively if the door and hinge assembly are to be shipped as one assembly, they may be welded together at the time of manufacture.) If shipped as three total pieces, the hinge assembly may be assembled to the autoclave body and then the door assembled to the hinge assembly, typically by use of a crane. Alternatively, the door may be first assembled to the hinge assembly, if not shipped as one piece, and then the combination assembled to the rest of the autoclave. In either case, the present invention can substantially reduce shipping costs for oversized and/or overweight autoclaves by reducing the size and weight of the autoclave body for shipping purposes, the shipment of two or three small/lighter assemblies being less expensive than the shipment of one, larger/heavier assembly.

The specific exemplary embodiment disclosed herein is approximately 13 feet in diameter, approximately 60 feet long and when assembled, weighs approximately 125,000 pounds. Of this, the hinge assembly weighs approximately 4000 pounds and the door weighs approximately 13,000 pounds. Thus the shipment of the autoclave body rather than the entire assembled autoclave reduces not only the size of the article being shipped, but also reduces its weight by approximately 14%. The cost reduction in shipping the autoclave body normally more than offsets the cost of shipping the other one or two assemblies by a substantial amount, though the parts may readily be assembled at their destination using equipment and personnel normally readily available at the destination.

Obviously certain specific embodiments have been disclosed herein for purposes of specificity, though other embodiments will readily be apparent to those skilled in the art. By way of example the box-like structures may be of some other design as desired. The box-like structures are a preferred structure however, from a simplicity in fabrication standpoint, from a structural standpoint and from a practical aesthetic standpoint. In particular the box-like structures are readily fabricated from steel plate. The vertical sides of the box-like structures absorb the vertical forces in pure shear, while the forces from the door being cantilevered (both when open and when closed) are absorbed by shear and tension and compression in the steel plates, making the suspension of the hinge assembly and the door from the autoclave body quite rigid. Of course internal ribbing, etc. may be included if desired.

Thus while certain preferred embodiments of the present invention have been disclosed herein, it will be obvious to those skilled in the art that various changes in form and detail may be made in the invention without departing from the spirit and scope of the invention as set out in the full scope of the following claims.

What is claimed is:

1. For assembling an autoclave having a cylindrical autoclave body having an axis, a door at one end of the autoclave body and a hinge assembly coupling the door to the autoclave body, an improvement comprising:
a hinge assembly attachment structure welded to the autoclave body adjacent the door end of the autoclave body;
a hinge assembly attachment structure welded to the door;
the hinge assembly having first and second hinge members rotatable with respect to each other about a hinge axis;
the first hinge member being boltable to the hinge assembly attachment structure welded to the autoclave body at positions closer to the autoclave axis than the hinge axis;
the second hinge member being boltable to the hinge assembly attachment structure welded to the door;
whereby the hinge assembly and the door may be shipped separate from the autoclave body, or the autoclave body, the hinge assembly and the door may be shipped as separate parts and assembled by bolting together at a destination.

2. The improvement of claim 1 wherein the hinge assembly attachment structure is a box-like structure welded at one end to the autoclave body and presenting at the other end, an outward facing first flange adjacent the autoclave body and circumscribing the box-like structure, the first flange having a plurality of bolt holes therein, the first hinge assembly having a box-like structure open at one side and circumscribed by a second flange, the second flange having a plurality of bolt holes cooperatively disposed with the bolt holes in the first flange.

3. The improvement of claim 2 wherein the first and second flanges are planar flanges.

4. The improvement of claim 3 wherein the axis of the autoclave body is parallel to the planar flanges.

5. An autoclave comprising:
a cylindrical autoclave body having an axis, the autoclave body having a hinge assembly attachment structure welded to the autoclave body adjacent a door end of the autoclave body defining a bolt-on attachment region in close proximity to an outer surface of the autoclave body;
an autoclave door having a door attachment structure welded thereto, the door attachment structure defining bolt-on attachment regions; and
a hinge assembly having first and second hinge members rotatable with respect to each other about a hinge axis, the first hinge member being bolted to the hinge assembly attachment structure welded to the autoclave body, the second hinge member being bolted to the hinge assembly attachment structure welded to the door;
whereby the hinge assembly and the door may be shipped separate from the autoclave body, or the autoclave body, the hinge assembly and the door may be shipped as separate parts, and assembled by bolting together at a destination.

6. The autoclave of claim 5 wherein the hinge assembly attachment structure is a box-like structure welded at one end to the autoclave body and presenting at the other end, an outward facing first flange adjacent the autoclave body and circumscribing the box-like structure, the first flange having a plurality of bolt holes therein, the first hinge assembly having a box-like structure open at one side and circumscribed by a second flange, the second flange having a plurality of bolt holes cooperatively disposed with the bolt holes in the first flange.

7. The improvement of claim 6 wherein the first and second flanges are planar flanges.

8. The improvement of claim 7 wherein the axis of the autoclave body is parallel to the planar flanges.

9. A method of manufacturing and shipping large autoclaves to reduce shipping costs comprising:
manufacturing an autoclave body having a first hinge attachment structure welded to a side of the autoclave body adjacent a door end of the autoclave body, the first hinge attachment structure having a provision for bolting a hinge member thereto adjacent the side of the autoclave;
manufacturing an autoclave door having a second hinge attachment structure welded thereto, the second hinge attachment structure having a provision for bolting a hinge member thereto adjacent the side of the autoclave;
manufacturing a hinge assembly having first and second hinge members rotatable with respect to each other about a hinge axis, the first hinge member having a provision for bolting to the first hinge attachment structure, the second hinge member having a provision for bolting to the second hinge attachment structure;
shipping the autoclave body as a unit, separate from the door and hinge assembly, to a destination, thereby reducing the shipping weight of the autoclave body by separately shipping the hinge assembly and door;
assembling the autoclave by bolting parts together at the destination.

10. The method of claim 9 wherein the door and hinge assembly are shipped as an assembly.

11. The method of claim 9 wherein the door and hinge assembly are shipped as separate parts.

12. The method of claim 9 wherein when the autoclave is assembled at the destination, the hinge axis is further outward from the side of the autoclave body than the first hinge attachment structure, whereby shipping the autoclave body as a unit to a destination, separate from the door and hinge assembly, also reduces the width of the autoclave body for shipping in comparison to shipping an assembled autoclave.

13. For assembling an autoclave having a cylindrical autoclave body having an axis, a door at one end of the autoclave body and a hinge assembly coupling the door to the autoclave body, an improvement comprising:
the hinge assembly having first and second hinge members rotatable with respect to each other about a hinge axis;
a hinge assembly attachment structure welded to the autoclave body adjacent the door end of the autoclave body;
the first hinge member being boltable to the hinge assembly attachment structure welded to the autoclave body at positions closer to the autoclave axis than the hinge axis;
the second hinge member being fastened to the door;
whereby the hinge assembly and the door may be shipped separate from the autoclave body and assembled to the autoclave body by bolting together at a destination.

14. The improvement of claim 13 wherein the hinge assembly attachment structure is a box-like structure welded at one end to the autoclave body and presenting at the other end, an outward facing first flange adjacent the autoclave body and circumscribing the box-like structure, the first flange having a plurality of bolt holes therein, the first hinge member having a box-like structure open at one side and circumscribed by a second flange, the second flange having a plurality of bolt holes cooperatively disposed with the bolt holes in the first flange.

15. The improvement of claim 14 wherein the first and second flanges are planar flanges.

16. The improvement of claim 15 wherein the axis of the autoclave body is parallel to the planar flanges.

17. For assembling an autoclave comprising:
a cylindrical autoclave body having an axis, the autoclave body having a hinge assembly attachment structure welded to the autoclave body adjacent a door end of the autoclave body defining a bolt-on attachment region in close proximity to an outer surface of the autoclave body;
an autoclave door; and
a hinge assembly having first and second hinge members rotatable with respect to each other about a hinge axis, the first hinge member being boltable to the hinge assembly attachment structure welded to the autoclave body, the second hinge member being fastened to the door;
whereby the hinge assembly and the door may be shipped separate from the autoclave body and assembled at a destination by bolting together.

18. The autoclave of claim 17 wherein the hinge assembly attachment structure is a box-like structure welded at one end to the autoclave body and presenting at the other end, an outward facing first flange adjacent the autoclave body and circumscribing the box-like structure, the first flange having a plurality of bolt holes therein, the first hinge assembly having a box-like structure open at one side and circumscribed by a second flange, the second flange having a plurality of bolt holes cooperatively disposed with the bolt holes in the first flange.

19. The improvement of claim 18 wherein the first and second flanges are planar flanges.

20. The improvement of claim 19 wherein the axis of the autoclave body is parallel to the planar flanges.

21. A method of manufacturing and shipping large autoclaves to reduce shipping costs comprising:
manufacturing an autoclave body having a first hinge attachment structure welded to a side of the autoclave body adjacent a door end of the autoclave body, the first hinge attachment structure having a provision for bolting a hinge member thereto adjacent the side of the autoclave;
manufacturing an autoclave door and a hinge assembly, the hinge assembly having first and second hinge members rotatable with respect to each other about a hinge axis, the first hinge member having a provision for bolting to the first hinge attachment structure, the second hinge member being attached to the door;
shipping the autoclave body as a unit, separate from the door and hinge assembly, to a destination, thereby reducing the shipping weight of the autoclave body by separately shipping the hinge assembly and door;
assembling the autoclave by bolting parts together at the destination.

22. The method of claim 21 wherein when the autoclave is assembled at the destination, the hinge axis is further outward from the side of the autoclave body than the first hinge attachment structure, whereby shipping the autoclave body as a unit to a destination, separate from the door and hinge assembly, also reduces the width of the autoclave body for shipping in comparison to shipping an assembled autoclave.
